(19) United States
(12) Patent Application Publication
(10) Pub. No.: US 2017/0232215 A1
(43) Pub. Date: Aug. 17, 2017

(54) ORAL AIRWAY

(71) Applicant: John Zannis, New Bern, NC (US)

(72) Inventor: John Zannis, New Bern, NC (US)

(21) Appl. No.: 15/041,227

(22) Filed: Feb. 11, 2016

(51) Int. Cl. A61M 16/04

(52) U.S. Cl. CPC A61M 16/0486; A61M 2202/0208; A61M 2205/33

(57) ABSTRACT

Present invention is a oral airway with a flexible air connector and tubing built into the trachea tube distal end. Additional access channels are also provided.

### COPYRIGHT NOTICE

A portion of the disclosure of this patent contains material that is subject to copyright protection. The copyright owner has no objection to the reproduction by anyone of the patent document or the patent disclosure as it appears in the Patent and Trademark Office patent files or records, but otherwise reserves all copyright rights whatsoever.

### BACKGROUND OF THE INVENTION

Field of the Invention

The present invention relates to a medical device. In particular, the present invention relates to an oral airway.

Description of Related Art

The oral airway, also known as an oropharyngeal airway, is a medical device utilized to maintain an open airway in a patient. The oral airway device is designed to prevent the patient's tongue from covering the epiglottis, which could prevent the patient from breathing during medical procedures. During a patient's unconscious periods, such as during surgery, the muscles in the jaw relax and can allow the tongue to obstruct the airway.

Generally, oral airways include an elongated and generally curved tube, having a distal and proximal end, wherein the proximal end is inserted into the mouth of the patient over the tongue. Once positioned, the device creates an air passageway between the mouth and the posterior pharyngeal wall. This prevents the tongue or epiglottis from falling backwards against the posterior pharynx, which happens especially when a patient is anesthetized. The oral airways are also used to introduce a number of different devices into the airway, such as breathing tubes, aiding the device in its passage down into the trachea. Because of this need to introduce devices with the oral airway, oral airways come in completely open designs and in designs where other devices are pre-molded into the oral airway. While there are numerous designs for an oral airway, they all have in common at least a partial opening at the distal end of the device. When oxygen is to be delivered during oral airway use, tubing or the like is inserted into the open airway proximal end and attached.

### BRIEF SUMMARY OF THE INVENTION

The present invention relates to the discovery that, when using an oral airway and just delivering oxygen, the previous designs of oral airways are inefficient and subject to movement, infections, and a host of other problems. The present invention is the discovery of an oral airway having oxygen tubing fused to the distal opening of the oral airway, with separate accessory openings in the distal end, resulting in a safer and more easy to use design.

Accordingly, in one embodiment of the present invention, there is an oral airway comprising a trachea tube, having a central tube and two side channels, the trachea tube having a proximal end for introducing air into the central channel and a distal end for introducing the oral airway into the trachea and delivering air to the trachea through the central channel wherein there is a flexible tube having a proximal and a distal end in sealed, fluid communication with the central channel at the proximal end of the of the trachea tube, and wherein the side channels begin at the proximal end of the trachea tube and end at the distal end of the trachea tube.

### BRIEF DESCRIPTION OF THE DRAWINGS

FIG. 1 is side view of an oral airway of the present invention.

FIG. 2 is a view of the flange of the present invention.

### DETAILED DESCRIPTION OF THE INVENTION

While this invention is susceptible to embodiment in many different forms, there is shown in the drawings, and will herein be described in detail, specific embodiments, with the understanding that the present disclosure of such embodiments is to be considered as an example of the principles and not intended to limit the invention to the specific embodiments shown and described. In the description below, like reference numerals are used to describe the same, similar or corresponding parts in the several views of the drawings. This detailed description defines the meaning of the terms used herein and specifically describes embodiments in order for those skilled in the art to practice the invention.

### Definitions

The terms “about” and “essentially” mean±10 percent.

The terms “a” or “an”, as used herein, are defined as one or as more than one. The term “plurality”, as used herein, is defined as two or as more than two. The term “another”, as used herein, is defined as at least a second or more. The terms “including” and/or “having”, as used herein, are defined as comprising (i.e., open language). The term “coupled”, as used herein, is defined as connected, although not necessarily directly, and not necessarily mechanically.

The term “comprising” is not intended to limit inventions to only claiming the present invention with such comprising language. Any invention using the term comprising could be separated into one or more claims using “consisting” or “consisting of” claim language and is so intended.

References throughout this document to “one embodiment”, “certain embodiments”, and “an embodiment” or similar terms mean that a particular feature, structure, or characteristic described in connection with the embodiment is included in at least one embodiment of the present invention. Thus, the appearances of such phrases in various places throughout this specification are not necessarily all referring to the same embodiment. Furthermore, the particular features, structures, or characteristics may be combined in any suitable manner in one or more embodiments without limitation.

The term “or” as used herein is to be interpreted as an inclusive or meaning any one or any combination. Therefore, “A, B or C” means any of the following: “A; B; C; A and B; A and C; B and C; A, B and C”. An exception to this definition will occur only when a combination of elements, functions, steps or acts are in some way inherently mutually exclusive.

The drawings featured in the figures are for the purpose of illustrating certain convenient embodiments of the present invention, and are not to be considered as limitations thereto. The term “means” preceding a present participle of an operation indicates a desired function for which there is one or more embodiments, i.e., one or more methods, devices, or apparatuses for achieving the desired function and that one skilled in the art could select from these or their equivalent in view of the disclosure herein and use of the term “means” is not intended to be limiting.

As used herein, the term “oral airway” refers to a device having a trachea tube for insertion into the mouth and pharynx/trachea of a patient to provide a breathing pathway. It will have a proximal end for introducing air into the central channel and other desired elements into the two side channels, and a distal end for introducing the oral airway into the trachea and delivering air and other things to the trachea. In particular, in this invention it is specifically for connecting to a source consisting of an air/oxygen source fused to the central channel without other tubes or connection devices to two separate channels. In all, there are three channels in the device. These oral airways are made of a rigid polymeric material that is sterilizable and has a hollow channel running from the proximal end to the distal end. In this device, there are two other channels, on either side of the hollow channel. There are 3 total channels (one larger central channel and two smaller side channels). The smaller side channels are open at the distal end. The larger central channel is fused to the oxygen delivery tubing at the flange.

As used herein, the term “tube” refers to a hollow airway, such as clear flexible plastic tubing used to transport oxygen in a medical situation. It is designed to deliver oxygen/air to the trachea, wherein the flexible tube has a proximal end and a distal end in sealed, fluid communication with the proximal end of the trachea tube without the possibility of becoming disconnected.

As used herein, the term “sealed fluid communication” refers to a tube, as described above, sealed or fused to the proximal end of the tracheal tube, such that the hollow channel of the tube is sealed to the hollow channel of the trachea tube.

As used herein, the term “oxygen tubing connector” refers to a device positioned at the proximal end of the tube for connecting to an oxygen source. Such connectors for connecting tubing are within the skill in the art.

As used herein, the term “air” refers to oxygen or another air source to air in the breathing of a patient. These include room air, concentrated oxygen, or 100% oxygen.

### DRAWINGS

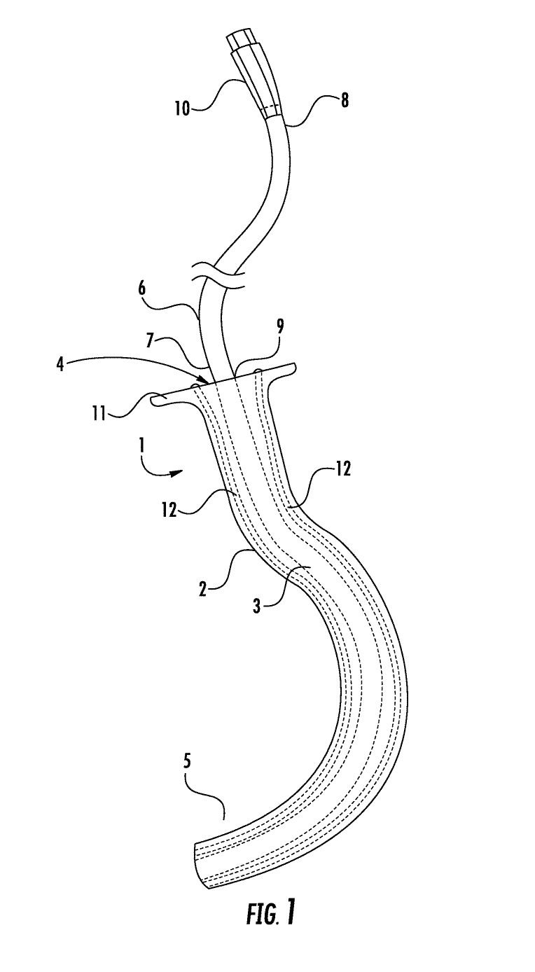

Now referring to the drawings, FIG. 1 is a perspective view of an oral airway of the present invention. Oral airway 1 consists of rigid polymeric trachea tube 2, having hollow center channel 3. It has a proximal end 4 and a distal end 5 for insertion into the mouth of a patient. A clear flexible tube 6 is attached, i.e. fused 9, to the proximal end 4 by the tubes distal end 7. The proximal tubing end 8 has a standard oxygen tubing connector 10. Also in this view is flange 11 which helps prevent inserting the trachea tube too far. Two side channels 12 are shown, which start at flange 11 and go to distal end 5 for insertion of other devices.

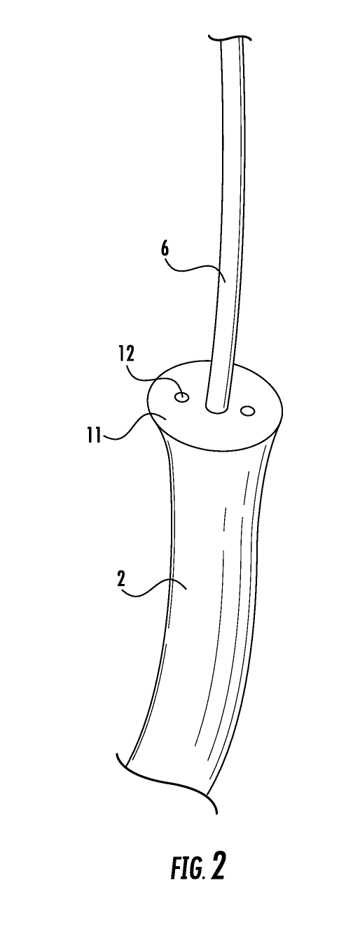

FIG. 2 is an angled top view of the intersection of the tube and the trachea tube. In this view, flange 11 has two side channel holes 12 used for inserting other devices within the channels and into the patient's trachea, used for insertion of smaller catheters (tubes) when needed. These would primarily be either a suction catheter used to remove oral secretions or a CO2 monitoring catheter used to calculate the amount of expired carbon dioxide when connected to such a monitoring device.

Those skilled in the art to which the present invention pertains may make modifications resulting in other embodiments employing principles of the present invention without departing from its spirit or characteristics, particularly upon considering the foregoing teachings. Accordingly, the described embodiments are to be considered in all respects only as illustrative, and not restrictive, and the scope of the present invention is, therefore, indicated by the appended claims rather than by the foregoing description or drawings. Consequently, while the present invention has been described with reference to particular embodiments, modifications of structure, sequence, materials and the like apparent to those skilled in the art still fall within the scope of the invention as claimed by the applicant.

What is claimed is:

1. An oral airway comprising a trachea tube, having a central tube and two side channels, the trachea tube having a proximal end for introducing air into the central channel and a distal end for introducing the oral airway into the trachea and delivering air to the trachea through the central channel wherein there is a flexible tube having a proximal and a distal end in sealed, fluid communication with the central channel at the proximal end of the of the trachea tube, and wherein the side channels begin at the proximal end of the trachea tube and end at the distal end of the trachea tube.

2. The oral airway according to claim 1 wherein there is an oxygen tubing connector attached to the proximal end of the flexible tubing.

3. The oral airway according to claim 1 wherein the flexible tubing is a clear polymeric tubing.

4. The oral airway according to claim 1 wherein the trachea tube is made of a rigid polymer.
